(19) United States
(12) Patent Application Publication
(10) Pub. No.: US 2026/0047808 A1
(43) Pub. Date: Feb. 19, 2026

(54) PHYSIOLOGICAL INFORMATION DISPLAY APPARATUS AND PHYSIOLOGICAL INFORMATION DISPLAY METHOD

(71) Applicant: NIHON KOHDEN CORPORATION, Tokyo (JP)

(72) Inventors: Yoshiharu HARADA, Tokorozawa-shi, Saitama (JP); Yoshihiro SUGO, Tokorozawa-shi, Saitama (JP); Sayaka YAMAZAKI, Tokorozawa-shi, Saitama (JP)

(73) Assignee: NIHON KOHDEN CORPORATION, Tokyo (JP)

(21) Appl. No.: 19/101,717

(22) PCT Filed: Aug. 4, 2023
(86) PCT No.: PCT/JP2023/028592

§ 371 Date: Feb. 6, 2025

(30) Foreign Application Priority Data
Aug. 10, 2022 (JP) 2022-127834

(51) Int. Cl. A61B 5/00; A61B 5/029

(52) U.S. Cl. CPC A61B 5/743; A61B 5/029; A61B 5/7235; A61B 5/7271

(57) ABSTRACT

A physiological information display apparatus includes a reception unit configured to receive a start signal and an end signal, the start signal indicating a start timing of a treatment for a subject, the end signal indicating an end timing of the treatment, a calculation unit configured to calculate a moving average value of a pulse wave transit time of the subject, a hemodynamic parameter of the subject, using the calculated moving average value, and a change rate of the hemodynamic parameter in a period from the start timing to the end timing, based on the start signal and the end signal received by the reception unit, and a display controller configured to output, to a display, the change rate calculated by the calculation unit.

### TECHNICAL FIELD

The present disclosure relates to a physiological information display apparatus and a physiological information display method.

In the related art, when performing a treatment such as administration of a fluid to a subject, a change in a parameter (hereinafter, referred to as a “hemodynamic parameter”) related to hemodynamics such as a stroke volume or a cardiac output of the subject is used as a useful parameter for checking a state of the subject. For example, a determination to administer a small amount of fluid, a determination to continue administration of the fluid if a change rate between a value of a hemodynamic parameter before the administration and a value of the hemodynamic parameter after the administration is equal to or greater than a certain value, a determination to end the administration to switch to another treatment if the change rate is less than the certain value are made. Further, JP-A-2005-312947 discloses a method of calculating a cardiac output using a pulse wave transit time.

### SUMMARY OF INVENTION

### Technical Problem

In order to check a change in hemodynamics before and after a treatment, for example, an operator who performs the treatment records a value of a hemodynamic parameter at the start of the treatment and a value of the hemodynamic parameter at the end of the treatment, and calculates using these values. For this reason, checking the change in hemodynamics before and after the treatment requires a lot of time and effort. Thus, there is a demand for a technique that can easily check a change in hemodynamics of a subject.

The present disclosure provides a physiological information display apparatus and a physiological information display method capable of easily checking a change in hemodynamics of a subject.

### Solution to Problem

A physiological information display apparatus includes:
a reception unit configured to receive a start signal and an end signal, the start signal indicating a start timing of a treatment for a subject, the end signal indicating an end timing of the treatment;
a calculation unit configured to calculate:
a moving average value of a pulse wave transit time of the subject;
a hemodynamic parameter of the subject, using the calculated moving average value; and
a change rate of the hemodynamic parameter in a period from the start timing to the end timing, based on the start signal and the end signal received by the reception unit; and
a display controller configured to output, to a display, the change rate calculated by the calculation unit.

A physiological information display method includes:
receiving a start signal indicating a start timing of a treatment for a subject;
receiving an end signal indicating an end timing of the treatment;
calculating:
a moving average value of a pulse wave transit time of the subject; and
a hemodynamic parameter of the subject, using the calculated moving average value;
calculating a change rate of the hemodynamic parameter in a period from the start timing to the end timing, based on the start signal and the end signal; and
outputting the calculated change rate to a display.

### Effects of Invention

According to the present disclosure, it is possible to easily check a change in hemodynamics of a subject.

### BRIEF DESCRIPTION OF DRAWINGS

FIG. 1 illustrates a configuration of a physiological information processing apparatus (physiological information display apparatus) according to the present disclosure.

FIG. 2 illustrates an example of a measurement mode using a monitor device that is an example of the physiological information processing apparatus of FIG. 1.

FIG. 3 illustrates an example of a screen displayed on a display of FIG. 1.

FIG. 4 illustrates an example of a screen displayed in a case where a treatment for a subject is newly started.

FIG. 5 is a diagram for illustrating an operation for instructing switching of a pulse wave transit time used in calculating a hemodynamic parameter.

FIG. 6 is a flowchart for illustrating operations of switching a pulse wave transit time used in calculating a hemodynamic parameter, in the physiological information processing apparatus according to an embodiment of the present disclosure.

### DESCRIPTION OF EMBODIMENTS

Exemplary embodiments of a physiological information display apparatus and a physiological information display method according to a presently disclosed subject matter will be described below with reference to the accompanying drawings.

### <Configuration of Physiological Information Processing Apparatus>

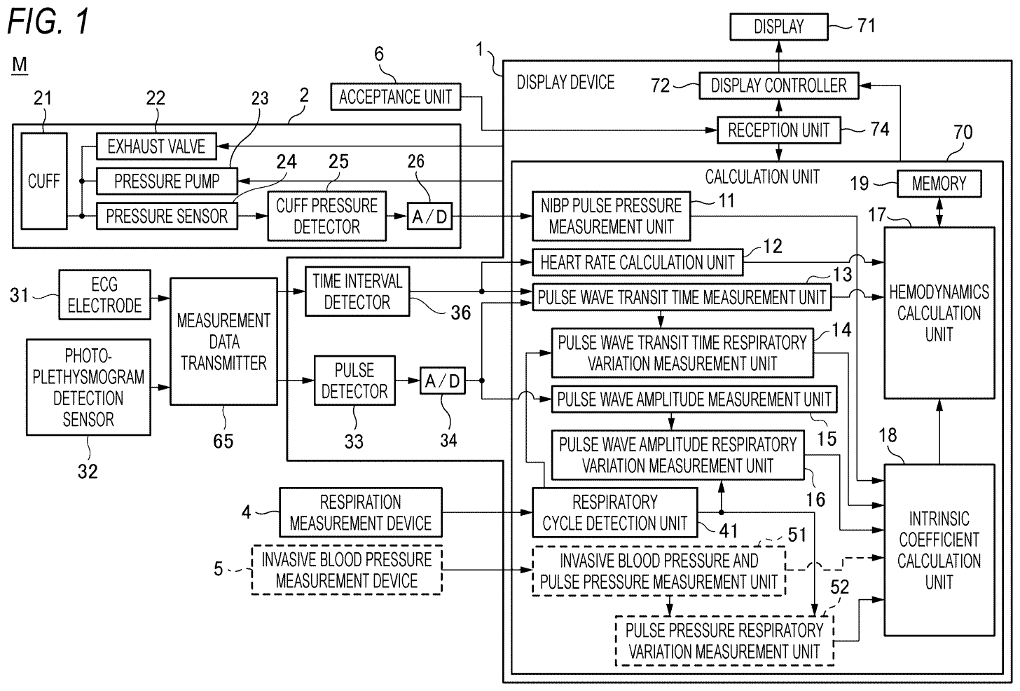

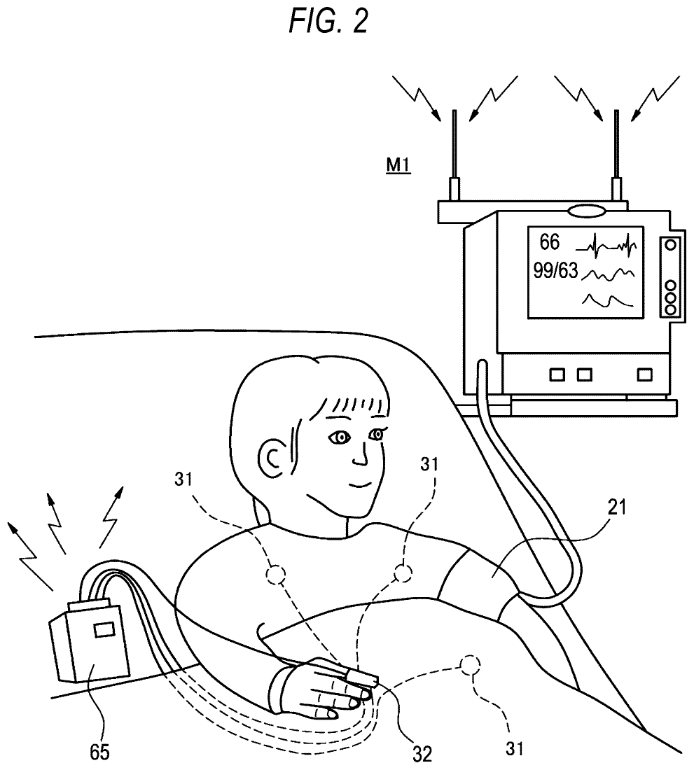

FIG. 1 illustrates a configuration of a physiological information processing apparatus (physiological information display apparatus) M according to the present disclosure. FIG. 2 illustrates an example of a measurement mode using a monitor device M1 that is an example of the physiological information processing apparatus M illustrated in FIG. 1.

The physiological information processing apparatus M may include a display device 1 configured to perform calculation, display control, and the like of a hemodynamic parameter related to hemodynamics of a subject, a blood pressure measurement device 2 configured to measure a systolic blood pressure and a diastolic blood pressure of the heart, a respiration measurement device 4, an invasive blood pressure measurement device 5, an acceptance unit 6, an ECG electrode 31, a photoplethysmogram detection sensor 32, a measurement data transmitter 65, and a display 71.

The blood pressure measurement device 2 is a device configured to measure a blood pressure of a subject by using a noninvasive blood pressure (NIBP) measurement method. The blood pressure measurement device 2 may include a cuff 21, an exhaust valve 22, a pressure pump 23, a pressure sensor 24, a cuff pressure detector 25, and an A/D converter 26. Specifically, as illustrated in FIG. 2, the blood pressure measurement device 2 is configured to measure a blood pressure with the cuff 21 attached to the upper arm of a subject.

An interior of the cuff 21 is configured to be opened or closed to the atmosphere by opening and closing the exhaust valve 22. The exhaust valve 22 is configured to be opened and closed based on, for example, a control signal output from the monitor device M1. The pressure pump 23 is configured to supply air to the cuff 21. The supply of air is controlled based on, for example, a control signal output from the monitor device M1.

The pressure sensor 24 is connected to the cuff 21. A sensor output of the pressure sensor 24 is detected by the cuff pressure detector 25. A sensor output from the cuff pressure detector 25 is converted into a digital signal by the A/D converter 26 and then input to an NIBP pulse pressure measurement unit 11 of the display device 1.

As illustrated in FIG. 2, the ECG electrode 31 is attached to the chest of the subject, and is configured to perform measurement with an R wave generation time point in an electrocardiogram serving as a reference point of a time interval. The ECG electrode 31 is electrically connected to the measurement data transmitter 65. Measurement data from the ECG electrode 31 is input to the measurement data transmitter 65 and then is wirelessly transmitted from the measurement data transmitter 65 to a time interval detector 36 in the display device 1 illustrated in FIG. 1.

As illustrated in FIG. 2, the photoplethysmogram detection sensor 32 is attached to a subject's periphery such as a finger. The photoplethysmogram detection sensor 32 is configured to measure, for example, a pulse wave in the periphery. A pulse wave transit time (PWTT) is obtained based on the measurement data obtained from the ECG electrode 31 and the pulse wave obtained from the photoplethysmogram detection sensor 32. The photoplethysmogram detection sensor 32 is electrically connected to the measurement data transmitter 65. Measurement data obtained by the photoplethysmogram detection sensor 32 is input to the measurement data transmitter 65, and is wirelessly transmitted from the measurement data transmitter 65 to a pulse detector 33 in the display device 1 illustrated in FIG. 1.

The respiration measurement device 4 is configured to continuously measure respiration of the subject. Measurement data obtained by measurement of the respiration measurement device 4 is input to a respiratory cycle detection unit 41 of the display device 1.

The invasive blood pressure measurement device 5 is configured to measure a blood pressure by an invasive blood pressure (IBP) method, that is, by inserting a catheter into a blood vessel of the subject. Measurement data obtained by the invasive blood pressure measurement device 5 is input to an invasive blood pressure and pulse pressure measurement unit 51 of the display device 1.

The acceptance unit 6 is configured to accept an input operation of the operator and is configured to generate an instruction signal corresponding to the input operation. The acceptance unit 6 is, for example, a touch panel disposed overlapping the display 71 described later, an operation button provided on a housing of the display device 1, or a mouse or keyboard connected to an input and output interface that is not illustrated (for example, a USB interface). The instruction signal generated by the acceptance unit 6 is input to the display device 1.

### <Configuration of Display Device>

The display device 1 may include the pulse detector 33, an A/D converter 34, the time interval detector 36, a calculation unit 70, a display controller 72, and a reception unit 74. The calculation unit 70 may include the NIBP pulse pressure measurement unit 11, a heart rate calculation unit 12, a pulse wave transit time measurement unit 13 (hereinafter PWTT measurement unit 13), a pulse wave transit time respiratory variation measurement unit 14 (hereinafter, PWTTRV measurement unit 14), a pulse wave amplitude measurement unit 15 (hereinafter, PWA measurement unit 15), a pulse wave amplitude respiratory variation measurement unit 16 (hereinafter, PWARV measurement unit 16), a hemodynamics calculation unit 17, an intrinsic coefficient calculation unit 18, a memory 19, the respiratory cycle detection unit 41, the invasive blood pressure and pulse pressure measurement unit 51, and a pulse pressure respiratory variation measurement unit 52 (hereinafter, PPRV measurement unit 52). The display device 1 may include a central processing unit (CPU), a read only memory (ROM), a random-access memory (RAM), a hard disk drive (HDD), and the like. The CPU may function as the calculation unit 70, the display controller 72, and the reception unit 74 and the like.

The time interval detector 36 is configured to acquire an ECG waveform, based on the measurement data that is received from the ECG electrode 31 via the measurement data transmitter 65. The time interval detector 36 is configured to convert the measurement data into a digital signal and is configured to output the digital signal to the heart rate calculation unit 12 and the PWTT measurement unit 13 of the calculation unit 70.

The pulse detector 33 is configured to acquire a waveform of a periphery in the photoplethysmogram, based on the measurement data that is received from the photoplethysmogram detection sensor 32 via the measurement data transmitter 65. Then, the pulse detector 33 is configured to output the measurement data to the A/D converter 34. The A/D converter 34 is configured to convert the measurement data into a digital signal and is configured to output the digital signal to the PWTT measurement unit 13 and the PWA measurement unit 15 of the calculation unit 70.

The NIBP pulse pressure measurement unit 11 is configured to measure an NIBP pulse pressure, based on blood pressure data obtained by measurement of the blood pressure measurement device 2. The NIBP pulse pressure is calculated based on a difference between a systolic (maximum) blood pressure value and a diastolic (minimum) blood pressure value. The measured NIBP pulse pressure is output to the intrinsic coefficient calculation unit 18.

The heart rate calculation unit 12 is configured to calculate the number of heart beats in one minute (heart rate, HR), based on the reference point (R wave generation time point) measured by the time interval detector 36. The calculated heart rate HR is input to the hemodynamics calculation unit 17.

The PWTT measurement unit 13 is configured to calculate the PWTT, which is a time lapsed from occurrence of the R wave to occurrence of a SpO₂ pulse wave in the electrocardiogram, based on the reference point (R wave generation time point) measured by the time interval detector 36 and the waveform of the periphery detected by the photoplethysmogram detection sensor 32.

More specifically, the PWTT measurement unit 13 is configured to calculate a moving average value of a plurality of PWTTs immediately before the current time point, in order to avoid disturbance of the value of the PWTT caused by the influence of instantaneous noise or the like. Specifically, in a case where the PWTT measurement unit 13 calculates 16 consecutive PWTTs, the PWTT measurement unit 13 calculates a moving average value of the 16 PWTTs. Hereinafter, the moving average value of the 16 PWTTs is referred to as a “moving average value PWTT-16”.

Further, in a case where the PWTT measurement unit 13 calculates the moving average value PWTT-16 for 4 consecutive times using 64 consecutive PWTTs, the PWTT measurement unit 13 calculates a moving average value of 4 moving average values PWTT-16. The moving average value of the PWTT calculated in this manner is referred to as a “moving average value PWTT-64”. Then, the PWTT measurement unit 13 is configured to output the calculated moving average value PWTT-64, as the PWTT, to the hemodynamics calculation unit 17 and the PWTTRV measurement unit 14.

The PWTTRV measurement unit 14 is configured to measure a respiratory variation of the PWTT, based on the PWTT calculated by the PWTT measurement unit 13 and the respiratory cycle detected by the respiratory cycle detection unit 41. Measurement data indicating the measured respiratory variation of the PWTT is input to the intrinsic coefficient calculation unit 18.

The PWA measurement unit 15 is configured to measure a pulse wave amplitude from the waveform of the periphery obtained by the pulse detector 33. The measured pulse wave amplitude is input to the PWARV measurement unit 16.

The respiratory cycle detection unit 41 is configured to detect a respiratory cycle, based on respiration data measured by the respiration measurement device 4. The detected respiratory cycle is input to the PWTTRV measurement unit 14, the PWARV measurement unit 16, and the PPRV measurement unit 52.

The PWARV measurement unit 16 is configured to measure a respiratory variation of the pulse wave amplitude (pulse amplitude variation, PAV), based on the pulse wave amplitude measured by the PWA measurement unit 15 and based on the respiratory cycle detected by the respiratory cycle detection unit 41. The measured respiratory variation of the pulse wave amplitude is input to the intrinsic coefficient calculation unit 18.

The invasive blood pressure and pulse pressure measurement unit 51 is configured to measure an IBP pulse pressure, based on blood pressure data measured by the invasive blood pressure measurement device 5. The measured IBP pulse pressure is input to the PPRV measurement unit 52.

The PPRV measurement unit 52 is configured to measure a respiratory variation of the pulse pressure (pulse pressure variation, PPV), based on the IBP pulse pressure measured by the invasive blood pressure and pulse pressure measurement unit 51 and based on the respiratory cycle measured by the respiratory cycle detection unit 41. Measurement data indicating the measured respiratory variation of the pulse pressure is input to the intrinsic coefficient calculation unit 18.

The intrinsic coefficient calculation unit 18 is configured to calculate coefficients intrinsic to the subject, based on the NIBP pulse pressure measured by the NIBP pulse pressure measurement unit 11, based on the respiratory variation of the PWTT measured by the PWTTRV measurement unit 14, based on the respiratory variation of the pulse wave amplitude measured by the PWARV measurement unit 16, and based on the respiratory variation of the pulse pressure measured by the PPRV measurement unit 52. The calculated coefficients are, for example, coefficients K, α, and β to be described later, and are input to the hemodynamics calculation unit 17.

Based on the heart rate HR calculated by the heart rate calculation unit 12, based on the PWTT measured by the PWTT measurement unit 13, and based on the coefficients K, α, and β calculated by the intrinsic coefficient calculation unit 18, the hemodynamics calculation unit 17 is configured to calculate a hemodynamic parameter of the subject. Here, it is assumed that the hemodynamics calculation unit 17 is configured to calculate, as the hemodynamic parameter, a flow rate (stroke volume, SV) of blood flowing into the aorta during a systolic phase of the heart and a cardiac output measured continuously in a noninvasive manner (noninvasive estimated continuous cardiac output, esCCO).

Further, the hemodynamics calculation unit 17 is configured to calculate a change rate of the hemodynamic parameter in a period (hereinafter, referred to as a “treatment period”) in which a treatment such as fluid administration to the subject or a treatment using a drug is performed. The calculation of the hemodynamic parameter and the calculation of the change rate of the hemodynamic parameter by the hemodynamics calculation unit 17 will be described later.

### <Calculation of Hemodynamic Parameter and Change Rate of Hemodynamic Parameter> (Calculation of Hemodynamic Parameter)

There is a correlation between stroke volume SV and PWTT as shown in Equation 1. In Equation 1, K, α, and β are coefficients intrinsic to the subject.

$$SV = K * ( α * PWTT + β ) \qquad ( Equation 1 )$$

The hemodynamics calculation unit 17 is configured to substitute the coefficients K, α, and β calculated by the intrinsic coefficient calculation unit 18 into Equation 1. The hemodynamics calculation unit 17 is configured to substitute, for example, the moving average value PWTT-64 received from the PWTT measurement unit 13 into the PWTT in Equation 1. Accordingly, the hemodynamics calculation unit 17 can calculate the stroke volume SV. The stroke volume SV calculated by the hemodynamics calculation unit 17 is hereinafter referred to as a “stroke volume esSV”.

The hemodynamics calculation unit 17 is configured to periodically calculate the stroke volume esSV, and is configured to store, in the memory 19, the calculated stroke volume esSV and a calculation timing of the calculated stroke volume esSV in association with each other, for example.

Further, in a case where the amount of blood pumped by beating of the heart (cardiac output, CO) is used, there is a correlation between stroke volume SV and heart rate HR as shown in

$$Equation 2$$

$$SV = CO / HR . \qquad ( Equation 2 )$$

By using Equation 1 and Equation 2, the noninvasive estimated continuous cardiac output esCCO can be calculated as indicated by the following Equation 3.

$$\begin{matrix}CO = SV * HR \\ = K * ( α * PWTT + β ) * HR \\ = esCCO\end{matrix} \qquad ( Equation 3 )$$

The hemodynamics calculation unit 17 is configured to substitute the coefficients K, a, and β into Equation 3. The hemodynamics calculation unit 17 is configured to substitute the moving average value PWTT-64 into the PWTT in Equation 3. Accordingly, the hemodynamics calculation unit 17 can calculate the noninvasive estimated continuous cardiac output esCCO.

The hemodynamics calculation unit 17 is configured to periodically calculate the noninvasive estimated continuous cardiac output esCCO, and is configured to store, in the memory 19, for example, the calculated noninvasive estimated continuous cardiac output esCCO and a calculation timing of the calculated noninvasive estimated continuous cardiac output esCCO in association with each other.

### <Calculation of Change Rate of Hemodynamic Parameter>

The operator who performs a treatment for the subject can input a start timing of the treatment to the physiological information processing apparatus M by performing a predetermined input operation on the acceptance unit 6. In a case where such an input operation is performed, the acceptance unit 6 is configured to output, to the display device 1, contents of the input operation and a start signal indicating a start timing.

The operator can input an end timing of the treatment to the physiological information processing apparatus M by performing a predetermined input operation on the acceptance unit 6. In a case where such an input operation is performed, the acceptance unit 6 is configured to output an end signal indicating an end timing to the display device 1.

Upon receiving the start signal or the end signal output from the acceptance unit 6, the reception unit 74 of the display device 1 is configured to output the received start signal or end signal to the calculation unit 70 and the display controller 72.

In a case where the start signal is received from the reception unit 74, the hemodynamics calculation unit 17 of the calculation unit 70 is configured to store, in the memory 19, the start timing indicated by the start signal. In a case where the end signal is received from the reception unit 74, the hemodynamics calculation unit 17 is configured to store, in the memory 19, the end timing indicated by the end signal.

After the end signal is received, the hemodynamics calculation unit 17 is configured to specify a treatment period based on the start timing and the end timing. Then, the hemodynamics calculation unit 17 is configured to calculate a change rate of the hemodynamic parameter in the specified treatment period.

For example, the hemodynamics calculation unit 17 is configured to refer to a plurality of noninvasive estimated continuous cardiac output esCCO stored in the memory 19, and is configured to specify a maximum value esCCOmax of noninvasive estimated continuous cardiac outputs esCCO calculated in the treatment period, and a minimum value esCCOmin of the noninvasive estimated continuous cardiac output esCCO calculated in the treatment period.

Then, by using the maximum value esCCOmax and the minimum value esCCOmin, the hemodynamics calculation unit 17 is configured to calculate a change rate of the noninvasive estimated continuous cardiac output esCCO as in the following Equation 4. The hemodynamics calculation unit 17 is configured to store the calculated change rate in the memory 19.

$$Change rate of esCCO = 2 * ( esCCO max - esCCO min ) / ( esCCO max + esCCO min ) \qquad ( Equation 4 )$$

In addition, the hemodynamics calculation unit 17 is configured to refer to a plurality of stroke volumes esSV stored in the memory 19, and is configured to specify a maximum value esSVmax of stroke volumes esSV calculated in the treatment period, and a minimum value esSVmin of the stroke volumes esSV calculated in the treatment period.

By using the maximum value esSVmax and the minimum value esSVmin, the hemodynamics calculation unit 17 is configured to calculate a change rate of the stroke volume esSV as in the following Equation 5. The hemodynamics calculation unit 17 is configured to store the calculated change rate in the memory 19.

$$Change rate of esSV = 2 * ( esSV max - esSV min ) / ( esSV max + esSV min ) \qquad ( Equation 5 )$$

The start signal from the acceptance unit 6 may be a signal that does not include information on the start timing. In this case, for example, the reception unit 74 is configured to notify the calculation unit 70 and the display controller 72 of a reception timing of the start signal as the start timing. It is the same or similar case for the end signal. In a case where the end signal does not include information on the end timing, for example, the reception unit 74 is configured to notify the calculation unit 70 and the display controller 72 of a reception timing of the end signal as the end timing.

### <Display Control Processing>

### (Display of Hemodynamic Parameter and Change Rate of Hemodynamic Parameter)

(a) Display During a Period in which No Treatment is Performed

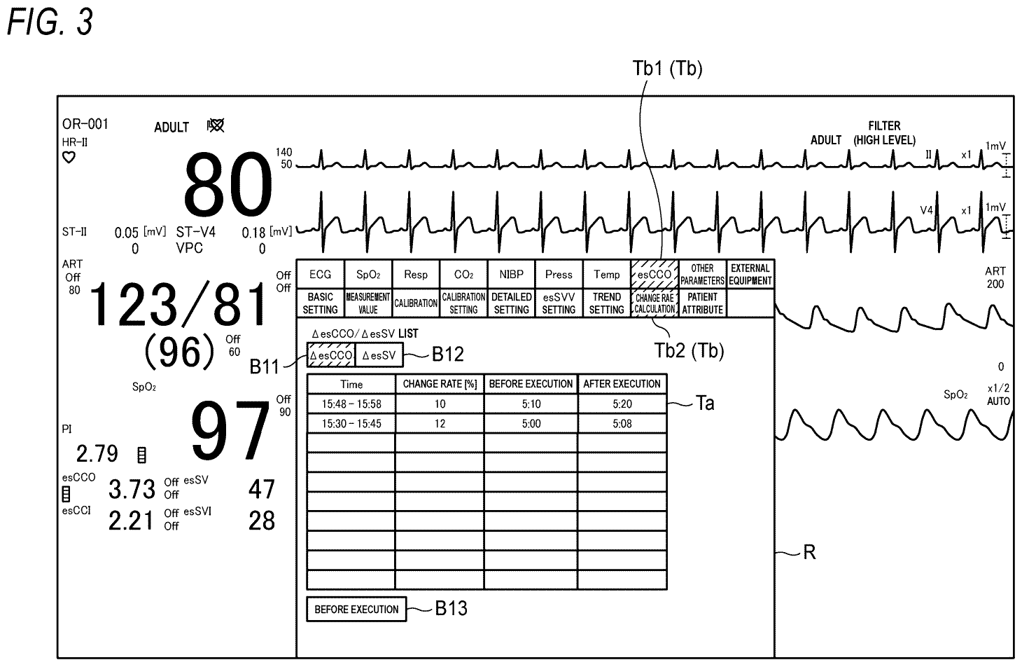

The display controller 72 is configured to output the hemodynamic parameter and the change rate of the hemodynamic parameter calculated by the hemodynamics calculation unit 17 to the display 71 such as a monitor. Accordingly, a screen including the hemodynamic parameter and the change rate of the hemodynamic parameter is displayed on the display 71. FIG. 3 illustrates an example of a screen displayed on the display 71 illustrated in FIG. 1.

As illustrated in FIG. 3, on the screen displayed on the display 71, the current heart rate, blood pressure value, noninvasive estimated continuous cardiac output esCCO, stroke volume esSV, and the like of the subject are displayed. In addition, the screen includes a region R in which a hemodynamic parameter in a treatment period of the subject is displayed.

The region R may include a plurality of tabs Tb. The plurality of tabs Tb include, for example, a tab Tb1 for selecting a display related to a hemodynamic parameter, and a tab Tb2 for selecting a display related to a change rate of the hemodynamic parameter. For example, letters “esCCO” are assigned to the tab Tb1. For example, letters “change rate calculation” are assigned to the tab Tb2.

FIG. 3 illustrates a screen displayed during a period in which no treatment is performed for the subject, and displayed in a case where the operator performs, on the acceptance unit 6 illustrated in FIG. 1, an input operation of selecting the tab Tb1 and further selecting the tab Tb2.

In a case where the input operation as described above is performed, the acceptance unit 6 is configured to output an instruction signal indicating contents of the input operation to the reception unit 74 of the display device 1. Upon receiving the instruction signal output from the acceptance unit 6, the reception unit 74 is configured to output the instruction signal to the display controller 72.

Upon receiving the instruction signal output from the reception unit 74, the display controller 72 is configured to read out the start timing and the end timing stored in the memory 19 and is configured to specify one or more treatment periods for the subject.

Further, the display controller 72 is configured to refer to a plurality of hemodynamic parameters and change rates of the plurality of hemodynamic parameters stored in the memory 19, and is configured to read out, from the memory 19, for each treatment period, a start timing, an end timing, a hemodynamic parameter associated with the start timing, a hemodynamic parameter associated with the end timing, and change rates of the hemodynamic parameters. Then, the display controller 72 is configured to perform control such that read-out values are displayed in the region R.

In addition, the display controller 72 is configured to switch the display of the change rate of the hemodynamic parameter between the display of the change rate of the noninvasive estimated continuous cardiac output esCCO, and the display of the change rate of the stroke volume esSV.

More specifically, the region R in a case where the tab Tb1 and the tab Tb2 are selected may include a selection button B11 for selecting the display of the noninvasive estimated continuous cardiac output esCCO, a selection button B12 for selecting the display of the stroke volume esSV, and a table Ta showing a list of change rates of a hemodynamic parameter for each treatment period. For example, letters and sign “ΔesCCO” are assigned to the selection button B11. For example, letters and sign “ΔesSV” are assigned to the selection button B12.

The operator can select any one of the selection buttons B11 and B12. In a case where neither the selection button B11 nor the selection button B12 is selected by the operator, the selection button B11 is automatically selected. That is, in such a state, the noninvasive estimated continuous cardiac output esCCO and the change rates of the noninvasive estimated continuous cardiac output esCCO are displayed in the table Ta.

Specifically, it is assumed that, for the subject, a first treatment is performed in a period from 15:30 to 15:45 and a second treatment is performed in a period from 15:48 to 15:58.

In this case, for example, it is displayed in the table Ta that the noninvasive estimated continuous cardiac output esCCO at 15:30 is 5.00, the noninvasive estimated continuous cardiac output esCCO at 15:45 is 5.08, and a change rate of the noninvasive estimated continuous cardiac output esCCO in the first treatment period is 12%. Further, it is displayed in the table Ta that the noninvasive estimated continuous cardiac output esCCO at 15:48 is 5.10, the noninvasive estimated continuous cardiac output esCCO at 15:58 is 5.20, and a change rate of the noninvasive estimated continuous cardiac output esCCO in the second treatment period is 10%.

In the table Ta, for example, a value in the latest treatment period is displayed in an upper row. Therefore, in a case where two treatments are performed, a value of the second treatment period is displayed in the first row, and a value in the first treatment period is displayed in the second row.

It is also assumed that the operator performs an input operation of selecting the selection button B12 included in the region R. In this case, the display controller 72 is configured to perform control such that the stroke volume esSV and change rates of the stroke volume esSV for each treatment period are displayed in the table Ta, instead of the noninvasive estimated continuous cardiac output esCCO and the change rates of the noninvasive estimated continuous cardiac output esCCO for each treatment period.

In a case where there is a change rate that is less than a predetermined threshold among the change rates of the hemodynamic parameter displayed in the table Ta, the display controller 72 may be configured to perform control such that the operator can easily recognize that the change rate of is less than the threshold. For example, the display controller 72 may be configured to perform control such that the change rate is displayed in a color different from that of the other change rates, or control such that a message indicating that the change rate is less than the threshold is displayed on the screen.

### (b) Display in Treatment Period

The region R may further include a selection button B13 for inputting a start timing and an end timing of a treatment. To the selection button B13, for example, letters “before execution” are assigned in a period in which no treatment is performed, and letters “after execution” are assigned in a treatment period.

As described above, the operator can input the start timing and the end timing of the treatment to the physiological information processing apparatus M by performing a predetermined input operation on the acceptance unit 6. The predetermined input operation is, for example, an operation of selecting the selection button B13 displayed in the region R.

That is, at the start timing of the treatment, the operator performs an operation of selecting the selection button B13 to which the letters “before execution” are assigned. Accordingly, the start timing is input to the physiological information processing apparatus M, and the letters of the selection button B13 are switched to “after execution”. Further, at the end timing of the treatment, the operator performs an operation of selecting the selection button B13 to which the letters “after execution” are assigned. Accordingly, the end timing is input to the physiological information processing apparatus M, and the letters of the selection button B13 are switched to “before execution”.

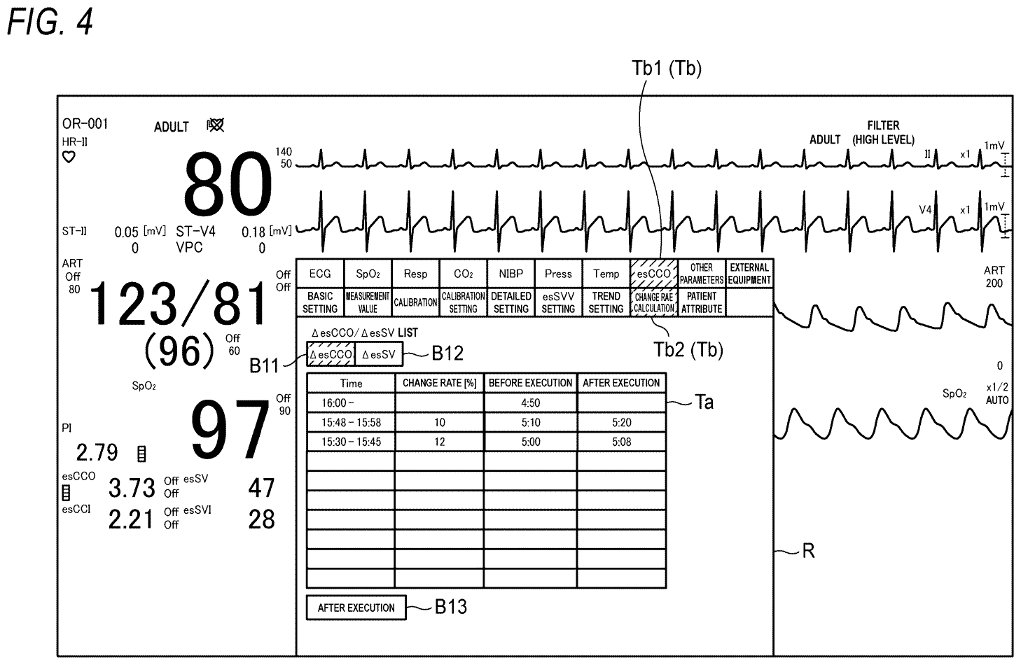

FIG. 4 illustrates an example of a screen displayed when a treatment for a subject is newly started. Referring to FIGS. 3 and 4, for example, it is assumed that the operator performs an input operation of selecting the selection button B13 illustrated in FIG. 3 on the acceptance unit 6, at 16:00, which is a timing at which the operator newly starts a treatment for the subject.

In this case, the acceptance unit 6 is configured to output an instruction signal, as a start signal, to the reception unit 74 of the display device 1, the instruction signal indicating contents of the input operation and the start timing of 16:00. Upon receiving the instruction signal output from the acceptance unit 6, the reception unit 74 is configured to output the instruction signal to the calculation unit 70 and the display controller 72.

Upon receiving the instruction signal output from the reception unit 74, the hemodynamics calculation unit 17 in the calculation unit 70 is configured to store, in the memory 19, as the start timing, the 16:00 indicated by the instruction signal.

Upon receiving the instruction signal output from the reception unit 74, the display controller 72 is configured to perform control such that the letters assigned to the selection button B13 on the screen are switched from “before execution” to “after execution”. The display controller 72 is configured to refer to a plurality of hemodynamic parameters stored in the memory 19 and is configured to read out a hemodynamic parameter corresponding to 16:00, which is the start timing, from the memory 19. Then, the display controller 72 is configured to perform control such that the start timing and a value of the read-out hemodynamic parameter are displayed in the table Ta.

Specifically, the display controller 72 is configured to perform control such that 16:00, which is the start timing of the latest treatment, and a value of the hemodynamic parameter at 16:00 are displayed in the first row of the table Ta. In addition, the display controller 72 is configured to perform control such that the values in the treatment period from 15:48 to 15:58 displayed in the first row in FIG. 3 are displayed in the second row, and the values in the treatment period from 15:30 to 15:45 displayed in the second row in FIG. 3 are displayed in a third row.

### (c) Display of Hemodynamic Parameter at Current Time Point

On the screen displayed on the display 71, as described above, the noninvasive estimated continuous cardiac output esCCO and the stroke volume esSV of the subject at the current time point are displayed. The display controller 72 is configured to periodically read out the latest noninvasive estimated continuous cardiac output esCCO and stroke volume esSV stored in the memory 19, and is configured to output, to the display 71, the read-out noninvasive estimated continuous cardiac output esCCO and stroke volume esSV.

In the screen illustrated in FIG. 4, as an example, “3.73” that is the noninvasive estimated continuous cardiac output esCCO at the current time point, and “47” that is the stroke volume esSV at the current time point are displayed.

In the screens illustrated in FIGS. 3 and 4, both the noninvasive estimated continuous cardiac output esCCO and the stroke volume esSV are displayed as the hemodynamic parameter of the subject at the current time point. Alternatively, one of the noninvasive estimated continuous cardiac output esCCO and the stroke volume esSV may be displayed.

### (Switching of Moving Average Value of PWTT Used in Calculating Hemodynamic Parameter)

Here, in a case where the heart rate HR is temporarily 80 bpm, it takes about one minute to calculate the moving average value PWTT-64 of 64 PWTTs. However, in a specific period such as a treatment period, it may be necessary to monitor a sudden change in the hemodynamics of the subject.

Therefore, for example, the physiological information processing apparatus M is configured to switch the moving average value of the PWTT used in calculating the hemodynamic parameter between the moving average value PWTT-64 of 64 PWTTs (first pulse wave transit times) and the moving average value PWTT-16 of 16 PWTTs (second pulse wave transit times).

### (a) Automatic Switching

Referring again to FIG. 1, the PWTT measurement unit 13 is configured to output the moving average value PWTT-64, as the PWTT, to the hemodynamics calculation unit 17, during a period in which no treatment is performed, that is, during a period in which no start signal is received from the reception unit 74. Accordingly, the hemodynamics calculation unit 17 is configured to calculate the hemodynamic parameter by using the moving average value PWTT-64 with high accuracy for which the influence of noise or the like is reduced.

That is, in a period in which no treatment is performed, a value using the moving average value PWTT-64 is displayed, on the display 71, as the current noninvasive estimated continuous cardiac output esCCO and stroke volume esSV. Therefore, the operator can check more accurate changes in the noninvasive estimated continuous cardiac output esCCO and the stroke volume esSV.

On the other hand, the PWTT measurement unit 13 is configured to output the moving average value PWTT-16, as the PWTT, to the hemodynamics calculation unit 17, during a treatment period, that is, a period during which no end signal is received from the reception unit 74 after the start signal is received from the reception unit 74. Accordingly, the hemodynamics calculation unit 17 is configured to calculate the hemodynamic parameter by using the moving average value PWTT-16 calculated at an early stage.

That is, in the treatment period, the current noninvasive estimated continuous cardiac output esCCO and stroke volume esSV are updated in the display 71 at a high frequency. Therefore, the operator can quickly check the change in the noninvasive estimated continuous cardiac output esCCO and the stroke volume esSV.

### (b) Manual Switching

The PWTT measurement unit 13 may be configured to switch the moving average value to be output to the hemodynamics calculation unit 17, as the PWTT, between the moving average value PWTT-64 and the moving average value PWTT-16 in a case where a predetermined input operation is performed by the operator, regardless of whether it is in the treatment period.

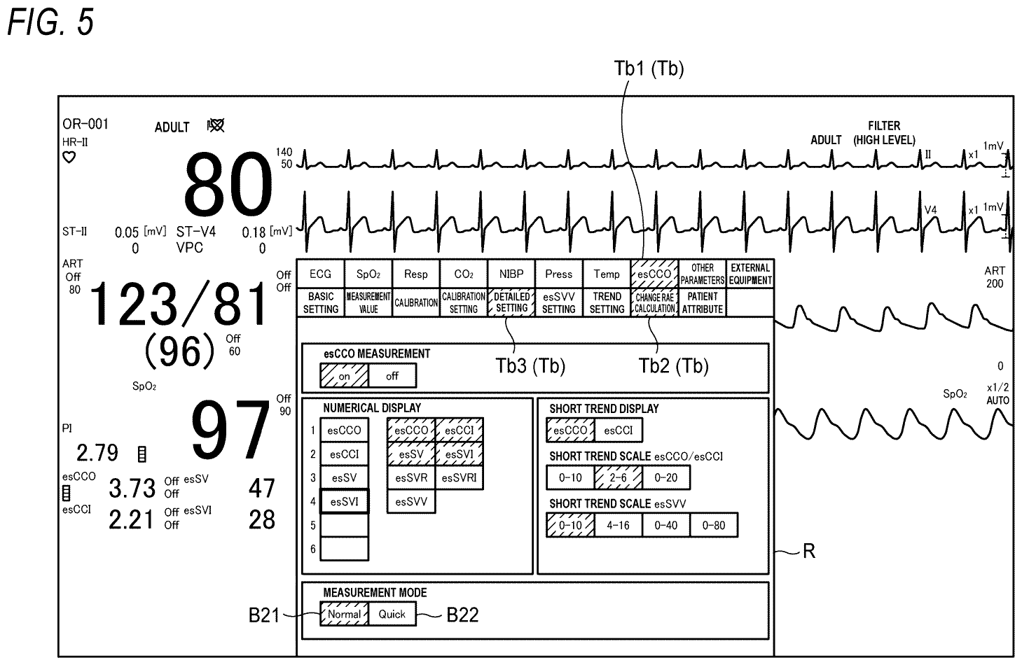

FIG. 5 is a diagram for illustrating an operation for instructing switching of the PWTT used in calculating the hemodynamic parameter. As illustrated in FIG. 5, the plurality of tabs Tb included in the region R include a tab Tb3 for selecting a display of the setting screen related to the hemodynamic parameter. For example, letters “Detailed Setting” are assigned to the tab Tb3.

FIG. 5 illustrates a screen displayed in a case where the operator performs, on the acceptance unit 6 illustrated in FIG. 1, an input operation of selecting the tab Tb1 and further selecting the tab Tb3. In a case where the input operation as described above is performed, the acceptance unit 6 is configured to output an instruction signal indicating contents of the input operation to the reception unit 74 of the display device 1. Upon receiving the instruction signal output from the acceptance unit 6, the reception unit 74 is configured to output the instruction signal to the display controller 72.

Upon receiving the instruction signal output from the reception unit 74, the display controller 72 is configured to perform control, based on the instruction signal, such that a setting screen related to the hemodynamic parameter is displayed on the display 71 as illustrated in FIG. 5. On the setting screen, selection buttons B21 and B22 for selecting the PWTT used in calculating the hemodynamic parameter are displayed. For example, letters “Normal” are assigned to the selection button B21. For example, letters “Quick” are assigned to the selection button B22.

It is assumed that the operator performs an input operation of selecting the selection button B21 on the acceptance unit 6. In a case where the input operation as described above is performed, the acceptance unit 6 is configured to output an instruction signal indicating contents of the input operation to the reception unit 74 of the display device 1. Upon receiving the instruction signal output from the acceptance unit 6, the reception unit 74 outputs the instruction signal to the calculation unit 70.

In a case where the instruction signal indicating that the selection button B21 is selected is received, the PWTT measurement unit 13 of the calculation unit 70 is configured to output the moving average value PWTT-64, as the PWTT, to the hemodynamics calculation unit 17. The hemodynamics calculation unit 17 is configured to calculate the hemodynamic parameter using the moving average value PWTT-64.

It is assumed that the operator performs an input operation of selecting the selection button B22 on the acceptance unit 6. In a case where the input operation as described above is performed, the acceptance unit 6 is configured to output an instruction signal indicating contents of the input operation to the reception unit 74 of the display device 1. Upon receiving the instruction signal output from the acceptance unit 6, the reception unit 74 outputs the instruction signal to the calculation unit 70.

In a case where the instruction signal indicating that the selection button B22 is selected is received, the PWTT measurement unit 13 of the calculation unit 70 is configured to switch the moving average value to be output to the hemodynamics calculation unit 17, as the PWTT, from the moving average value PWTT-64 to the moving average value PWTT-16. Then, the hemodynamics calculation unit 17 is configured to calculate the hemodynamic parameter using the moving average value PWTT-16.

In a case where neither the selection button B21 nor the selection button B22 is selected by the operator, the selection button B21 is automatically selected. Therefore, in such a state, the hemodynamics calculation unit 17 is configured to calculate the hemodynamics parameter using the moving average value PWTT-64.

The moving average value of the PWTT that can be used to calculate the hemodynamic parameter is not limited to being switchable between the two types of the moving average value PWTT-16 and the moving average value PWTT-64, and may be switchable between three or more types of moving average values.

In addition, in a case where the tab Tb2 for selecting the display related to the change rate of the hemodynamic parameter is selected, the PWTT measurement unit 13 may be configured to switch the moving average value to be output to the hemodynamics calculation unit 17 from the moving average value PWTT-64 to the moving average value PWTT-16.

A physiological information display apparatus including the hemodynamics calculation unit 17, the intrinsic coefficient calculation unit 18 and the memory 19 of the calculation unit 70, the display 71, the display controller 72, and the reception unit 74, may be provided separately from a processing apparatus including the other components of the calculation unit 70.

The display 71 may be provided inside the display device 1. In a case where the physiological information display apparatus is provided separately from the processing apparatus as described above, the display 71 may be provided inside the physiological information display apparatus.

### <Flowchart of Operations>

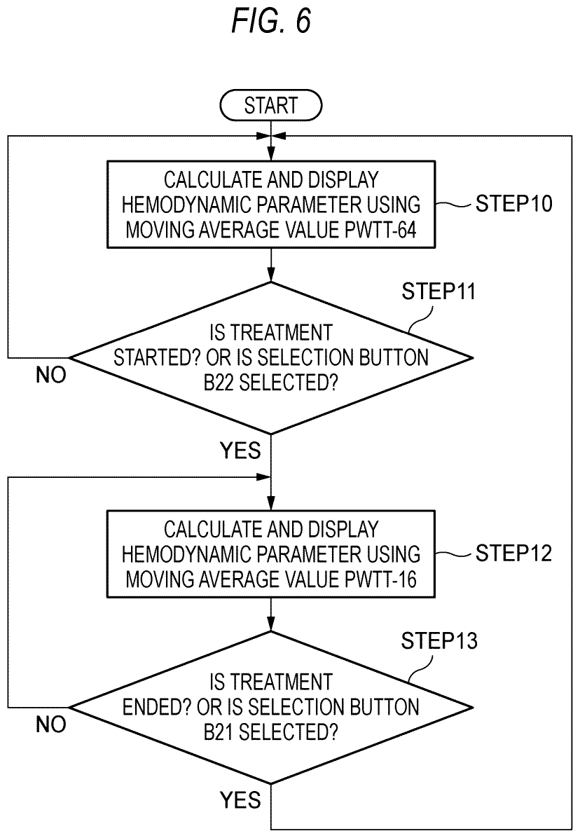

FIG. 6 is a flowchart for illustrating operations of switching the PWTT used in calculating the hemodynamic parameter in the physiological information processing apparatus M according to the embodiment of the present disclosure.

Referring to FIG. 6, first, for example, in a case where the operator activates the display device 1, each unit in the calculation unit 70 performs measurement or the like. At this time, the PWTT measurement unit 13 outputs the moving average value PWTT-64, as the PWTT, to the hemodynamics calculation unit 17, and the hemodynamics calculation unit 17 calculates a hemodynamic parameter using the moving average value PWTT-64. Then, the hemodynamic parameter calculated by the hemodynamics calculation unit 17 is displayed on the display 71 (STEP 10).

Next, in a case where a treatment for the subject is not started, that is, in a case where an input operation of a start timing of the treatment is not performed by the operator, or in a case where an operation of selecting the selection button B22 of FIG. 5 is not performed by the operator (“NO” in STEP 11), the operation shown in STEP 10 is continuously performed.

On the other hand, it is assumed that the treatment for the subject is started, that is, the input operation of the start timing of the treatment is performed by the operator, or assumed that the operation of selecting the selection button B22 illustrated in FIG. 5 is performed by the operator (“YES” in STEP 11). In this case, the PWTT measurement unit 13 switches the moving average value to be output to the hemodynamics calculation unit 17, as the PWTT, from the moving average value PWTT-64 to the moving average value PWTT-16. Then, the hemodynamics calculation unit 17 calculates the hemodynamic parameter using the moving average value PWTT-16, and the display 71 displays the hemodynamic parameter calculated by the hemodynamics calculation unit 17 (STEP 12).

Next, in a case where the treatment for the subject is not ended, that is, in a case where an input operation of an end timing of the treatment is not performed by the operator, or in a case where an operation of selecting the selection button B21 of FIG. 5 is not performed by the operator (“NO” in STEP 13), the operation shown in STEP 12 is continuously performed.

On the other hand, in a case where the treatment for the subject is ended, that is, in a case where the input operation of the end timing of the treatment is performed by the operator, or in a case where the operation of selecting the selection button B21 of FIG. 5 is performed by the operator (“YES” in STEP 13), the operation shown in STEP 10 is performed again. The operations from STEP 10 to STEP 13 are repeated, for example, until the operator stops the physiological information processing apparatus M.

As described above, in the physiological information processing apparatus M according to an aspect of the present disclosure, the reception unit 74 is configured to receive a start signal indicating a start timing of a treatment for a subject and an end signal indicating an end timing of the treatment. The calculation unit 70 is configured to calculate a moving average value of the PWTT of the subject, is configured to calculate a hemodynamic parameter of the subject using the calculated moving average value, and is further configured to calculate a change rate of the hemodynamic parameter in a treatment period from the start timing to the end timing based on the start signal and the end signal received by the reception unit 74. The display controller 72 is configured to perform control of outputting the calculated change rate to the display 71.

As described above, with the configuration in which the change rate of the hemodynamic parameter is automatically calculated and displayed, it is possible to visually and easily check the change in the hemodynamics of the subject during the treatment period for the subject. In addition, by calculating the hemodynamic parameter using the moving average value of a plurality of PWTTs, it is possible to display a more accurate value avoiding the influence of noise or the like.

In the physiological information processing apparatus M according to another aspect of the present disclosure, the display controller 72 is configured to switch the display of the change rate between a display of a change rate of the stroke volume (esSV) and a display of a change rate of the noninvasive estimated continuous cardiac output (esCCO). With such a configuration, the operator can freely select and check the change rate of the esSV and the change rate of the esCCO.

In the physiological information processing apparatus M according to another aspect of the present disclosure, the display controller 72 is further configured to perform control of outputting, to the display 71, a hemodynamic parameter calculated by the calculation unit 70 using a moving average value of a plurality of PWTTs immediately before a current time point. With such a configuration, not only the change rate of the hemodynamic parameter in a treatment period but also the hemodynamic parameter can be quickly checked.

In the physiological information processing apparatus M according to another aspect of the present disclosure, the display controller 72 is further configured to perform control of outputting, to the display 71, the start timing of treatment, the end timing, a hemodynamic parameter calculated using a moving average value of a plurality of PWTTs immediately before the start timing, and a hemodynamic parameter calculated using a moving average value of a plurality of PWTTs immediately before the end timing. With such a configuration, it is possible to check the hemodynamic parameters at the start timing and the end timing of the treatment for the subject.

In the physiological information processing apparatus M according to another aspect of the present disclosure, the display controller 72 is configured to perform control of outputting a list of the change rates of a plurality of times of the treatments to the display 71, based on the start signal and the end signal received by the reception unit 74. With such a configuration, it is possible to compare changes in the hemodynamics in a plurality of times of treatments.

In the physiological information processing apparatus M according to another aspect of the present disclosure, the calculation unit 70 is configured to calculate the moving average value of the PWTT of the subject and is configured to calculate the hemodynamic parameter of the subject using the calculated moving average value. The calculation unit 70 is configured to switch a calculation target between a hemodynamic parameter using the moving average value PWTT-64 for the first PWTTs, and a hemodynamic parameter using the moving average value PWTT-16 for the second PWTTs that are shorter than the first PWTTs.

With such a configuration, for example, in a situation where a change in the hemodynamics of the subject should be checked quickly, the hemodynamic parameter is calculated using the moving average value PWTT-16 of the second PWTTs, and in a situation where a highly accurate change in the hemodynamics for which the influence of noise or the like is reduced should be checked, the hemodynamic parameter can be calculated using the moving average value PWTT-64 of the first PWTTs. Accordingly, physiological information of the subject can be processed by a more appropriate method according to a use state.

In the physiological information processing apparatus M according to another aspect of the present disclosure, the reception unit 74 is configured to receive a start signal indicating a start timing of a treatment for a subject and an end signal indicating an end timing of the treatment. Based on the start signal and the end signal received by the reception unit 74, the calculation unit 70 is configured to calculate the hemodynamic parameter using the moving average value PWTT-64 in a situation where no treatment is performed, and is configured to calculate the hemodynamic parameter using the moving average value PWTT-16 in the treatment period.

As described above, in the period in which the treatment is performed for the subject, the change in the hemodynamics can be more quickly checked by calculating the hemodynamic parameter using the moving average value PWTT-16 of the second PWTTs calculated at an early stage. On the other hand, in a situation where no treatment is performed, a more accurate change in the hemodynamics can be checked by calculating the hemodynamic parameter using the highly accurate moving average value PWTT-64 of the first PWTTs for which the influence of noise or the like is reduced.

In the physiological information processing apparatus M according to another aspect of the present disclosure, the calculation unit 70 is configured to switch a calculation target from the hemodynamic parameter using the moving average value PWTT-64 to the hemodynamic parameter using the moving average value PWTT-16, in a case where the acceptance unit 6, which accepts an operation of an operator, receives a predetermined operation. With such a configuration, the calculation target can be switched between the hemodynamic parameter using the moving average value PWTT-64 and the hemodynamic parameter using the moving average value PWTT-16 at any timing of the operator.

Although the embodiments of the present disclosure have been described above, the technical scope of the present application should not be construed as being limited to the description of the embodiments. The embodiments are merely an example, and it is understood by those skilled in the art that various modifications of the embodiments are possible within the scope of the inventions described in the claims. The technical scope of the present application should be determined based on the scope of the inventions described in the claims and equivalents thereof.

This application claims priority to Japanese Patent Application No. 2022-127834 filed on Aug. 10, 2022, the entire content of which is incorporated herein by reference.

### INDUSTRIAL APPLICABILITY

According to the present disclosure, it is possible to provide a physiological information display apparatus and a physiological information display method capable of easily checking a change in hemodynamics of a subject.

1. A physiological information display apparatus comprising:
a reception unit configured to receive a start signal and an end signal, the start signal indicating a start timing of a treatment for a subject, the end signal indicating an end timing of the treatment;
one or more processors configured to calculate:
a moving average value of a pulse wave transit time of the subject;
a hemodynamic parameter of the subject, using the calculated moving average value; and
a change rate of the hemodynamic parameter in a period from the start timing to the end timing, based on the start signal and the end signal received by the reception unit; and
a display controller configured to output, to a display, the change rate calculated by the one or more processors.

2. The physiological information display apparatus according to claim 1,
wherein the display controller is configured to switch a display of the change rate between a display of a change rate of a stroke volume and a display of a change rate of a cardiac output.

3. The physiological information display apparatus according to claim 1,
wherein the display controller is further configured to output the hemodynamic parameter to the display, the hemodynamic parameter being calculated, by the one or more processors, using the moving average value of a plurality of pulse wave transit times immediately before a current time point.

4. The physiological information display apparatus according to claim 1,
wherein the display controller is further configured to output to the display:
the start timing;
the end timing;
the hemodynamic parameter calculated, by the one or more processors, using the moving average value of a plurality of pulse wave transit times immediately before the start timing; and
the hemodynamic parameter calculated, by the one or more processors, using the moving average value of a plurality of pulse wave transit times immediately before the end timing.

5. The physiological information display apparatus according to claim 1,
wherein the display controller is configured to output, to the display, a list of change rates of a plurality of times of the treatments, based on the start signal and the end signal received by the reception unit.

6. A physiological information display method comprising:
receiving a start signal indicating a start timing of a treatment for a subject;
receiving an end signal indicating an end timing of the treatment;
calculating:
a moving average value of a pulse wave transit time of the subject; and
a hemodynamic parameter of the subject, using the calculated moving average value;
calculating a change rate of the hemodynamic parameter in a period from the start timing to the end timing, based on the start signal and the end signal; and
outputting the calculated change rate to a display.
